(19) United States
(12) Patent Application Publication
(10) Pub. No.: US 2009/0010097 A1
(43) Pub. Date: Jan. 8, 2009

(54) System and Method for Heat Treating a Homogenized Fluid Product

(76) Inventor: Oleg V. Kozyuk, North Rldgeville, OH (US)

Correspondence Address: BENESCH, FRIEDLANDER, COPLAN & ARONOFF LLP;ATTN: IP DEPARTMENT DOCKET CLERK, 2300 BP TOWER, 200 PUBLIC SQUARE, CLEVELAND, OH, 44114, US

(21) Appl. No.: 12/208,570

(22) Filed: Sep. 11, 2008

Related U.S. Application Data
(63) Continuation of application No. 10/743,450, filed on Dec. 19, 2003, now abandoned.

(51) Int. Cl. B01F 15/06

(52) U.S. Cl. 366/147

(57) ABSTRACT

A system and method for heat treating a homogenized fluid product, the method comprising the steps of feeding a stream of fluid product ingredients through a local constriction of flow to effectuate high shear mixing of the fluid product ingredients in a high shear mixing zone downstream from the local constriction of flow and thereby form a homogenized fluid product at a first temperature and introducing a sufficient amount of the homogenized fluid product at a second temperature, which is less than the first temperature, into the high shear mixing zone to effectuate mixing of the homogenized fluid product at the first temperature with the homogenized fluid product at the second temperature to thereby heat treat the homogenized fluid product fluid product.

### BACKGROUND

The present invention relates to a system and method for heat treating a homogenized fluid product. The present invention has applicability in the food, beverage, pharmaceutical biotechnology, semiconductor, paint, ink, toner, fuel, magnetic media, and cosmetic industries.

Many different types of fluid products are heat treated, either through heating and/or cooling, during the production process. For example, during a pasteurization process, a fluid product such as a fruit juice is heated for a sufficient amount of time and at a sufficient temperature to kill all or substantially all of the microorganisms initially present in the liquid.

In another example, during a homogenization process, two or more fluid product ingredients can be subjected to shear forces, impact forces, and/or cavitation to form a homogenized fluid product. The shear forces, impact forces, and/or cavitation can cause a significant increase in temperature of the resultant fluid product. If one or more of the fluid product ingredients is a temperature sensitive material such as biological, organic, pharmaceutical, cellular, microbial, plant extracts, animal extracts, and certain food materials, the homogenized fluid product should be quickly cooled to prevent damage to the temperature sensitive material. Otherwise, the temperature sensitive material may be destroyed and wasted.

There are several methods known in the art to cool a homogenized fluid product. One such method is to introduce a cooling liquid such as water or a cooling agent to the fluid product. Such a method can reduce the temperature of the fluid product post-homogenization; however, the cooling liquid must be separated from the fluid product at a later stage in the process.

Another method to cool a homogenized fluid product is to introduce a compressed gas such as air or nitrogen to the homogenized fluid product. Once again, such a method can reduce the temperature of the fluid product post-homogenization; however, the compressed gas must be separated from the fluid product at a later stage in the process. Also, the compressed gas can react with the fluid product ingredients.

Yet another method to cool a homogenized fluid product is to introduce a cooled liquid that is the same liquid as one of the fluid product ingredients. This method results in a change in concentration with respect to the liquid.

Yet another method to cool a homogenized fluid product is to pass the fluid product through a heat exchanger to remove the heat from the fluid product. Although this method can reduce the temperature of the fluid product, it often takes a significant amount of time to cool the product to the desired fluid product temperature causing a loss in fluid product.

### BRIEF DESCRIPTION OF THE DRAWINGS

It will be appreciated that the illustrated boundaries of elements (e.g., boxes or groups of boxes) in the figures represent one example of the boundaries. One of ordinary skill in the art will appreciate that one element may be designed as multiple elements or that multiple elements may be designed as one element. An element shown as an internal component of another element may be implemented as an external component and vice versa.

Further, in the accompanying drawings and description that follow, like parts are indicated throughout the drawings and description with the same reference numerals, respectively. The figures are not drawn to scale and the proportions of certain parts have been exaggerated for convenience of illustration.

FIG. 1 is a schematic diagram of one embodiment of a system 100 for heat treating a homogenized fluid product;

FIG. 2 illustrates one embodiment of a high shear mixing device 200 that can be used in the system 100 of FIG. 1;

FIG. 3 illustrates one embodiment of a high shear mixing device 300 that can be used in the system 100 of FIG. 1;

FIG. 4 illustrates one embodiment of a high shear mixing device 400 that can be used in the system 100 of FIG. 1;

FIG. 5 illustrates one embodiment of a high shear mixing device 500 that can be used in the system 100 of FIG. 1;

FIG. 6 is a schematic diagram of another embodiment of a system 600 for heat treating a homogenized fluid product;

FIG. 7 illustrates one embodiment of a methodology for heat treating a homogenized fluid product; and

FIG. 8 illustrates another embodiment of a methodology for heat treating a homogenized fluid product.

### DETAILED DESCRIPTION OF ILLUSTRATED EMBODIMENTS

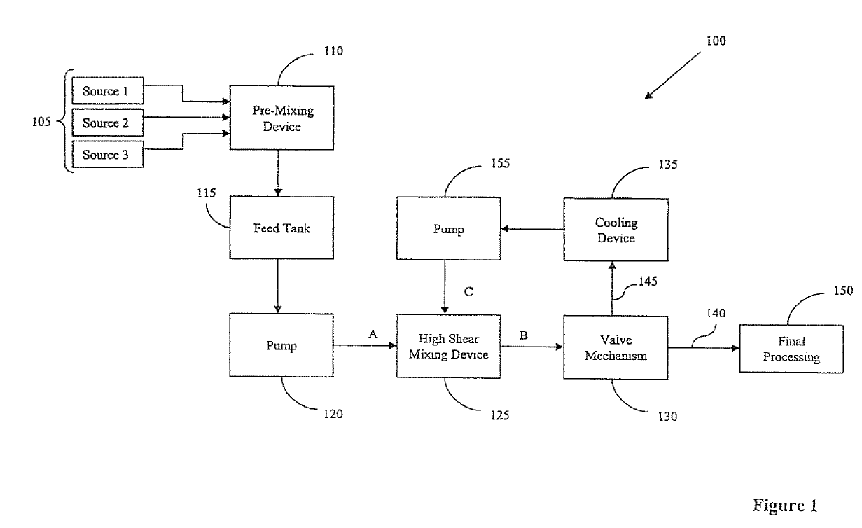

Illustrated in FIG. 1 is one embodiment of a system 100 for heat treating a homogenized fluid product. The system 100 can be practiced to heat treat many different types of fluid products such as pure liquid products, emulsions, liquid products carrying particles (e.g., suspensions), or liquid-gas dispersions. Fluid products may be produced for diverse uses such as food, beverages, pharmaceuticals, paints, inks, toners, fuels, magnetic media, and cosmetics. In one embodiment, the fluid product can be used in the food, pharmaceutical, and biotechnology industries and includes temperature sensitive material(s) that can be damaged and/or destroyed due to prolonged heating.

As shown in FIG. 1, the system 100 generally includes a feed tank 115 for storing pre-mixed fluid product ingredients, a high shear mixing device 125 fluidly coupled to the feed tank 115 and being configured to process the pre-mixed fluid product ingredients into a homogenized fluid product, and a valve mechanism 130 fluidly coupled to the high shear mixing device 125. As explained in further detail below, the valve mechanism 130 can direct a portion of the homogenized fluid product exiting the high shear mixing device 125 along a primary flow path 140 and a remaining portion of the homogenized fluid product along a cooling flow path 145. The portion of the homogenized fluid product following the primary flow path 140 can be directed to one or more final processing stages.

The cooling flow path 145 can include a cooling device 135 fluidly coupled between the valve mechanism 130 and the high shear mixing device 125. The cooling device 135 can be configured to cool the homogenized fluid product before it is returned to the high shear mixing device 125. For example, the remaining portion of the homogenized fluid product following the cooling flow path 145 can be directed back to the high shear mixing device 125 and used as a cooling fluid for heat treating the newly homogenized fluid product to prevent damage to any temperature sensitive material present in the fluid product due to prolonged heating. By returning a portion of the homogenized fluid product (at a lower temperature) to the high shear mixing device 125 for heat treating the homogenized fluid product, there is little or no change in the concentration of the heat treated fluid product. Also, there is no need for separation of the cooling fluid since the cooling fluid is the homogenized fluid product at a lower temperature.

With further reference to FIG. 1, one embodiment of the system 100 can include one or more sources 105 of fluid product ingredients or components. The fluid product sources 105 can be any type of storage container or tank capable of storing the fluid product ingredients. For simplicity, only three sources 105 of product ingredients are illustrated in FIG. 1 by way of example, but it will be appreciated that more or less fluid product ingredients could be used depending on the fluid product to be made. Fluid product ingredients can include, for example, liquids, solids, additives, gases, etc.

With further reference to FIG. 1, the fluid product ingredients can be supplied from the sources 105 into a pre-mixing device 110. The pre-mixing device 110 can be any suitable mixing device (e.g., propeller mixer, colloid mill, etc) depending on the fluid product ingredients being mixed. After pre-mixing, the fluid product ingredients can then be led into a feed tank 115. Optionally, the pre-mixing of the fluid product ingredients may be performed inside the feed tank 115.

The pre-mixed fluid product ingredients in the feed tank 115 can be supplied in the form of a stream to the high shear mixing device 125 via a pump 120. The pump 120 may be any type of pump normally used for the fluid product, provided it can generate the required feed pressure for proper operation of the high shear mixing device 125. In high pressure applications, a positive displacement pump such as a triplex or intensifier pump can be used.

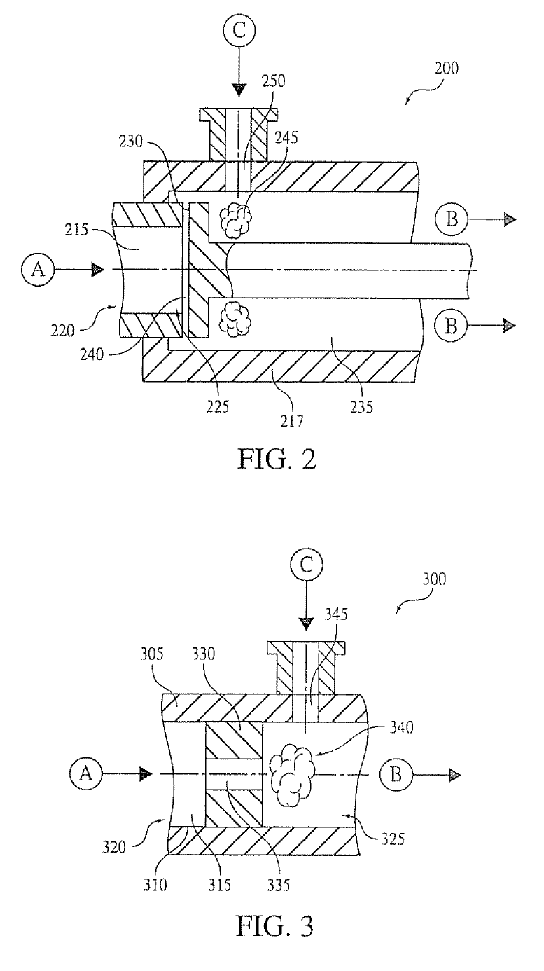

As discussed above, the high shear mixing device 125 can be configured to process the pre-mixed ingredients to form a homogenized fluid product. Examples of suitable high shear mixing devices include, but are not limited to, homogenizers, hydrodynamic cavitation mixing devices, other static mixers and flow reactors, and jet meels. FIGS. 2-5 illustrate several exemplary high shear mixing devices, which will be discussed in further detail below.

In one embodiment, the high shear mixing device 125 can be configured to include a local constriction of flow (not shown) where fluid product ingredients are forced under pressure through such local constriction of flow to effectuate high shear mixing of the fluid product ingredients in a high shear mixing zone (not shown) downstream from the local constriction of flow and thereby form a homogenized fluid product. Depending on the conditions (e.g., pressure and flow rate of the fluid stream and size and shape of the local constriction of flow), the fluid product ingredients may be subjected to not only high shear forces, but also impact forces and cavitation in the high shear mixing zone. The high shear mixing device 125 can also include a port (not shown) or other type of opening to permit introduction of a second fluid stream into the high shear mixing zone to effectuate mixing of the homogenized fluid product with the second fluid stream.

In another embodiment, the high shear mixing device 125 can be configured to permit introduction of at least two fluid streams (each including at least one fluid product ingredient) into a passageway (not shown) for impingement mixing of the fluid streams in a high shear mixing zone (not shown) and thereby form a homogenized fluid product. Depending on the conditions (e.g., pressure and flow rate of the fluid streams and the interaction between the fluid streams), the fluid product ingredients may be subjected to not only high shear forces, but also impact forces and cavitation in the high shear mixing zone. The high shear mixing device 125 can also include a port (not shown) or other type of opening to permit introduction of a third fluid stream into the high shear mixing zone to effectuate mixing of the homogenized fluid product with the third fluid stream.

Due to the shear forces, impact forces, and/or cavitation generated in the high shear mixing zone, the fluid product typically exits the high shear mixing device 125 at a temperature T2, which is greater than the input temperature of the fluid stream T1. For example, the temperature of water can increase about 30° C. after being passed through a local constriction of flow having a pressure drop of 20,000 psi (i.e., increase about 1° C.-2° C. for every 1000 psi of pressure drop through the local constriction of flow). It will be appreciated that temperature increase may vary depending on the viscosity and density of the particular fluid, the concentration of the ingredients, and the geometry of the local constriction of flow. This increase in temperature can cause problems when the fluid product includes a temperature sensitive material that is used in the food, pharmaceutical, and biotechnology industries. For example, certain temperature sensitive materials can be damaged and/or destroyed if they reach a certain critical temperature.

To prevent damage and/or destruction of the temperature sensitive material present in the homogenized fluid product, the homogenized fluid product at temperature T2 can be cooled to a desired fluid product temperature, which is typically less than the critical temperature of the temperature sensitive material present in the homogenized fluid product. As explained in further detail below, the homogenized fluid product at temperature T2 can be cooled by mixing it with substantially the same homogenized fluid product at a temperature that is less than T2.

With further reference back to FIG. 1, the fluid product typically exits the high shear mixing device 125 and enters the valve mechanism 130. The valve mechanism 130 can direct a portion of the homogenized fluid product exiting the high shear mixing device 125 along a primary flow path 140 and a remaining portion of the homogenized fluid product at temperature T2 along a cooling flow path 145. The valve mechanism 130 can be a manually-operated valve mechanism or a computer-controlled valve mechanism. Suitable valve mechanisms can include a two-way valve, a manifold system, or other fluid distribution system. In one embodiment, the selective portion of the fluid product directed along the cooling flow path 145 can be predetermined based on the viscosities and densities of the fluid product ingredients and the temperature, flow rate, and pressures of the fluid stream.

Along the primary flow path 140, the homogenized fluid product can be directed to one or more final processing stages. For example, the primary flow path 140 can be in fluid communication with a container filling device 150, such as an apparatus capable of filling bottles or cans with the fluid product. The container filling device 150 may optionally include a bowl shaped reservoir for temporarily storing the liquid product. Alternatively, the homogenized fluid product flowing through the primary flow path 140 could be processed further and/or stored in a large container or tank (not shown).

Along the cooling flow path 145, the homogenized fluid product can pass through the cooling device 135. The cooling device 135 can be configured to cool the fluid product to a temperature T3, which can be less than temperature T2, For example, the difference in temperature between temperature T3 and temperature T2 can be at least about 10° C. However, it will be appreciated that the difference in temperature between temperature T3 and temperature T2 can be at least about 1° C. depending on the homogenized fluid product being processed and the ingredients included therein. Alternatively, the difference in temperature between temperature T3 and temperature T2 can be at least about 1%, Examples of suitable cooling devices that can be used include, but are not limited to, a refrigerant-based cooling device, a shell and tube heat exchanger, or any other known heat exchange design.

The homogenized fluid product at temperature T3 can then be returned back to the high shear mixing device 125 via pump 155, which can be similar to the pump 120 discussed above, and introduced into the high shear mixing zone for intimate mixing with the newly homogenized fluid product at temperature T2. The mixing of the cooled homogenized fluid product at temperature T3 with the newly homogenized fluid product at temperature 12 can heal treat the homogenized fluid product to the desired fluid product temperature. Additionally, because the cooled homogenized fluid product at T3 is introduced into the high shear mixing zone for mixing with the newly homogenized fluid product at temperature T2, the mixing conditions can be improved resulting in rapid heat treatment of the homogenized fluid product to the desired fluid product temperature. For example, the homogenized fluid product at temperature T2 can be cooled virtually instantaneously (e.g., within as little as a few microseconds) to minimize and/or prevent damage the temperature sensitive material present in the fluid product.

Once the system 100 is in operation and the valve mechanism 130 is directing appropriate portions of the homogenized fluid product along both the primary and cooling flow paths 140, 145, the homogenized fluid product can exit the high shear mixing device 125 at the desired fluid product temperature. However, it will be appreciated that to maintain the temperature of the homogenized fluid product exiting the high shear mixing device 125 at the desired fluid product temperature, an adequate amount of the homogenized fluid product exiling the high shear mixing device 125 at the desired fluid product temperature should still be directed along the cooling flow path 145. This should ensure that an adequate supply of the cooled homogenized fluid product will be directed back to the high shear mixing device for mixing with the newly homogenized fluid product.

Optionally, to optimize the process, the system 100 may include temperature sensors provided; 1) at the inlet of the high shear mixing device 125 to detect the temperature T1 of the pre-mixed fluid product ingredients before they enter the high shear mixing device 125; 2) directly after the local constriction of flow or in the high shear mixing zone to detect the temperature T2 of the homogenized fluid product before it mixes with the cooled homogenized fluid product; 3) at the outlet of the cooling device 135 to detect the temperature T3 of the cooled homogenized fluid product; and 4) at the outlet of the high shear mixing device 125 to detect the temperature T4 of the homogenized fluid product exiting the high shear mixing device 125. Also, the system can optionally include flow meters provided at the inlet of the high shear mixing device 125 to detect the flow rate of the stream of pre-mixed ingredients before they enter the high shear mixing device 125 and at the outlet of the cooling device 135 to detect the flow rate of the cooled homogenized fluid product.

Optionally, the system 100 can further include a controller (not shown) including one or more microprocessors that can be used to regulate the temperature of the fluid product cooled in the cooling device 135. The controller can also be used control other components in the system 100, such as the pumps to regulate the pressure and flow rate of the fluid streams.

FIG. 2 illustrates a cross-sectional view of one embodiment of a high shear mixing device 200 that can be used in the system 100. The device 200 is essentially a fixed-gap type homogenizer shown and described in U.S. Pat. No. 4,944,602, which is hereby incorporated by reference in its entirety herein. The device 200 includes a flow-through channel or chamber 215. The flow-through channel 215 can further include an inlet 220 configured to introduce a fluid stream into the device 200 along a path represented by arrow A and an outlet 225.

The device 200 can further include a plate 230 provided in a chamber 235 downstream from the outlet 225 of the flow-through channel 215 thereby producing a gap therebetween (i.e., a local constriction 240 of flow). The local constriction 240 of flow can be configured to generate a high shear mixing zone 245 downstream from the local constriction 240 of flow and thereby form a resultant fluid product that exits the device 200 along a path represented by arrow B.

With further reference to FIG. 2, the flow-through channel 215 can further include a port 250 for introducing a second fluid stream into the flow-through channel 215 along a path represented by arrow C. In one embodiment, the port 250 can be disposed in the chamber 235 downstream from the local constriction 240 of flow to permit the introduction of the second fluid stream into the high shear mixing zone 245. It will be appreciated that any number of ports can be provided in the chamber 235 to introduce multiple fluid streams into the high shear mixing zone 245

FIG. 3 illustrates a cross-sectional view of one embodiment of a high shear mixing device 300 that can be used in the system 100. The device 300 is essentially a orifice-type hydrodynamic caviatation device shown and described in U.S. Pat. No. 5,969,207, which is hereby incorporated by reference in its entirety herein. The device 300 includes a wall 305 having an inner surface 310 that defines a flow-through channel or chamber 315. The flow-through channel 315 can further include an inlet 320 configured to introduce a fluid stream into the device 300 along a path represented by arrow A and an outlet 325 configured to exit the resultant fluid product from the device 300 along a path represented by arrow B.

The device 300 can further include a cavitation generator that generates high shear forces and/or hydrodynamic cavitation downstream from the cavitation generator. For example, the device 300 can include a cavitation generator that can include a plate 330 having an orifice 335 disposed therein to produce a local constriction of flow, it will be appreciated that the plate can be embodied as a disk when the flow-through channel 315 has a circular cross-section, or each plate can be embodied in a variety of shapes and configurations that can match the cross-section of the flow-through channel 315. To vary the degree and character of the cavitation field generated downstream from the plate 330, the orifice 335 can be embodied in a variety of different shapes and configurations. It will be appreciated that the orifice 335 can be configured in the shape of a Venturi tube, nozzle, orifice of any desired shape, or slot. Further, it will be appreciated that the orifice 335 can be embodied in other shapes and con figurations such as the ones disclosed in U.S. Pat. No. 5,969,207. In this embodiment, the orifice 335 disposed in the plate 330 can be configured to generate a high shear forces and/or hydrodynamic cavitation in a zone 340 downstream from the orifice 335

With further reference to FIG. 3, the flow-through channel 315 can further include a port 345 for introducing a second fluid stream into the flow-through channel 315 along a path represented by arrow C. In one embodiment, the port 345 can be disposed in the wall 305 downstream from the local constriction 340 of flow to permit the introduction of the second fluid stream into the mixing zone 340. It will be appreciated that any number of ports can be provided in the wall 305 to introduce multiple fluid streams into the mixing zone 340.

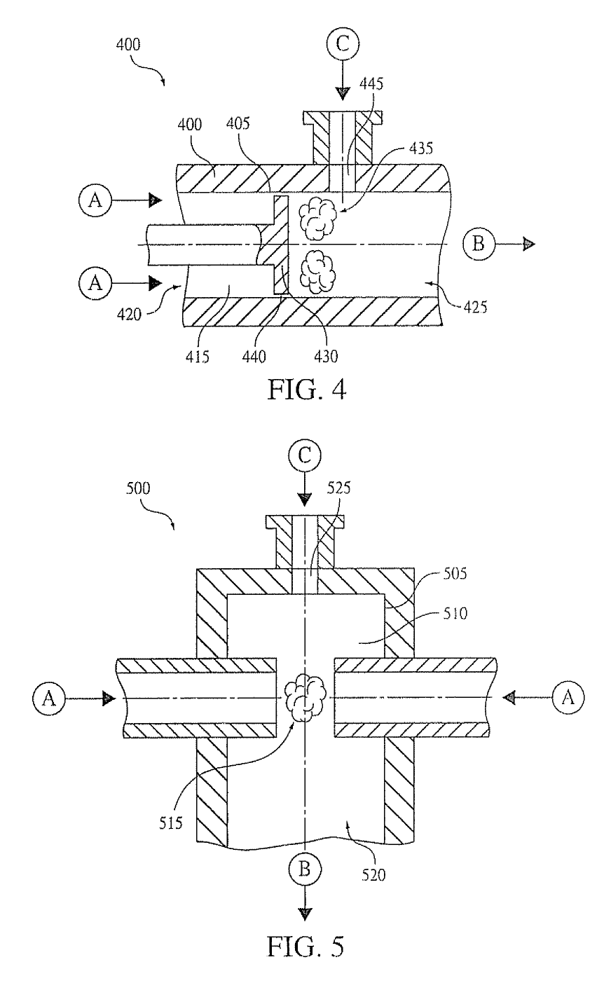

FIG. 4 illustrates a cross-sectional view of one embodiment of a high shear mixing device 400 that can be used in the system 100. The device 400 is essentially a baffle-type hydrodynamic caviatation device shown and described in U.S. Pat. No. 5,969,207. The high shear mixing device 400 includes a wall 405 having an inner surface 410 that defines a flow-through channel or chamber 415. The flow-through channel 415 can further include an inlet 420 configured to introduce a fluid stream into the device 400 along a path represented by arrow A and an outlet 425 configured to exit the resultant fluid product from the device 400 along a path represented by arrow B.

The device 400 can further include a cavitation generator that generates high shear forces and/or hydrodynamic cavitation downstream from the cavitation generator. For example, the device 400 can include a cavitation generator, such as a disc-shaped baffle 430. To vary the degree and character of the cavitation fields generated downstream from the baffle 430, the baffle 430 can be embodied in a variety of different shapes and configurations. It will be appreciated that the baffle 430 can be embodied in other shapes and configurations such as the ones disclosed in U.S. Pat. No. 5,969,207. In this embodiment, the baffle 430 can be configured to generate a high shear forces and/or hydrodynamic cavitation in a mixing zone 435 downstream from the baffle 430 via a local constriction 440 of fluid flow. For example, the local constriction 440 of liquid flow can be an area defined between the inner surface 410 of the wall 405 and an outer surface of the baffle 430.

With further reference to FIG. 4, the flow-through channel 415 can further include a port 445 for introducing a second fluid stream into the flow-through channel 415 along a path represented by arrow C. In one embodiment, the port 445 can be disposed in the wall 405 downstream from the local constriction 440 of flow to permit the introduction of the second fluid stream into the mixing zone 435. It will be appreciated that any number of ports can be provided in the wall 405 to introduce multiple fluid streams into the mixing zone 435.

FIG. 5 illustrates a cross-sectional view of one embodiment of a high shear mixing device 500 that can be used in the system 100. The device 500 is essentially a classic fluid impingement device shown and described in U.S. Pat. No. 2,751,335, which is hereby incorporated by reference in its entirety herein.

The device 500 includes a housing 505 defining a passageway 510 configured to permit introduction of at least two fluid streams, represented by arrows A, therein through openings 512 for impingement mixing thereof. The impingement of the two fluid streams can generate high shear forces, impact forces, and/or hydrodynamic cavitation in a mixing zone 515 in the passageway 510. The device 500 can further include an outlet 520 configured to exit the resultant fluid product from the device 500 along a path represented by arrow B.

In one embodiment, the housing 505 can further include a port 525 for introducing a third fluid stream into the passageway 510 along a path represented by arrow C. In one embodiment, the port 525 can be disposed in the housing 505 to permit the introduction of the second fluid stream into the mixing zone 515. It will be appreciated that any number of posts can be provided in the wall 505 to introduce multiple fluid streams into the mixing zone 515.

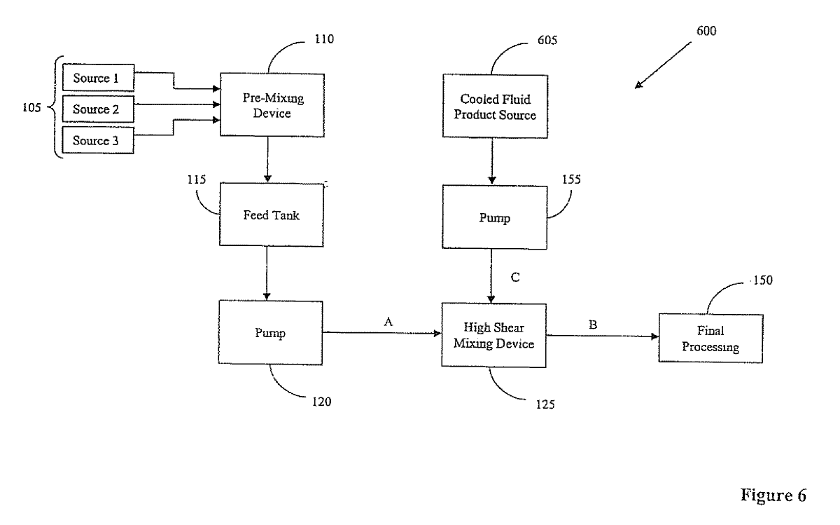

Illustrated in FIG. 6 is another embodiment of a system 600 for heat treating a homogenized fluid product. The system 600 can include similar components and operate in a similar manner to the system 100, except that the system 600 lacks the cooling flow path 145 of the system 100, Instead, the system 600 can include a separate source 605 of the homogenized fluid product that is stored at temperature T3. The homogenized fluid product at temperature T3 is substantially the same fluid product as the fluid product at temperature T2 (i.e., having substantially the same components and concentration levels).

Like the system 100 discussed above, the system 600 can be configured to permit the homogenized fluid product at temperature T3 to be supplied to the high shear mixing device 125 via pump 155 and introduced into the high shear mixing zone for intimate mixing with the newly homogenized fluid product fluid product at temperature T2. The mixing of the cooled homogenized fluid product at temperature T3 with the newly homogenized fluid product at temperature T2 can heat treat the homogenized fluid product to the desired fluid product temperature. Additionally, because the cooled homogenized fluid product at T3 is introduced into the high shear mixing zone for mixing with the newly homogenized fluid product at temperature T2, the mixing conditions can be improved resulting in rapid heat treatment of the homogenized fluid product to the desired fluid product temperature.

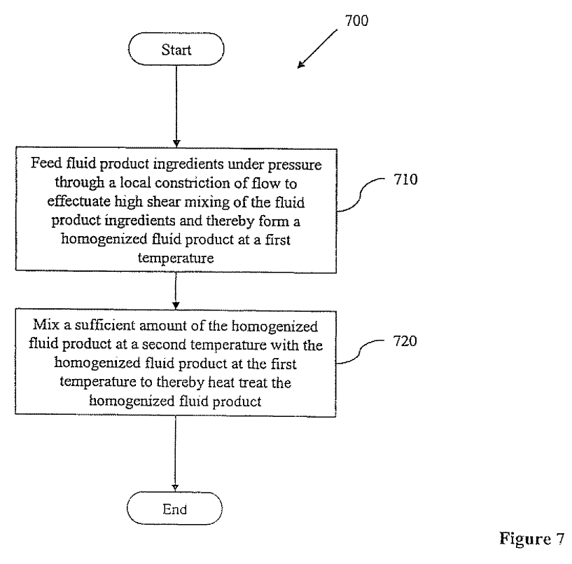

Illustrated in FIG. 7 is one embodiment of a methodology associated with heat treating a fluid product. The illustrated elements denote “processing blocks” and represent functions and/or actions taken for heat treating a fluid product. In one embodiment, the processing blocks may represent computer software instructions or groups of instructions that cause a computer or processor to perform an action(s) and/or to make decisions that control another device or machine to perform the processing. It will be appreciated that the methodology may involve dynamic and flexible processes such that the illustrated blocks can be performed in other sequences different than the one shown and/or blocks may be combined or, separated into multiple components. The foregoing applies to all methodologies described herein.

With reference to FIG. 7, the process 700 includes feeding fluid product ingredients under pressure through a local constriction of flow to effectuate high shear mixing of the fluid product ingredients and thereby form a fluid product at a first temperature (block 710). The high shear mixing of the fluid product ingredients can take place in, for example, a high shear mixing zone downstream from the local constriction of flow. A sufficient amount of the fluid product at a second temperature can then be mixed with the fluid product at the first temperature to thereby heat treat the fluid product (block 720). In one embodiment, the second temperature can be less than the first temperature resulting in the cooling of the fluid product when the fluid product at a second temperature is mixed with the fluid product at the first temperature.

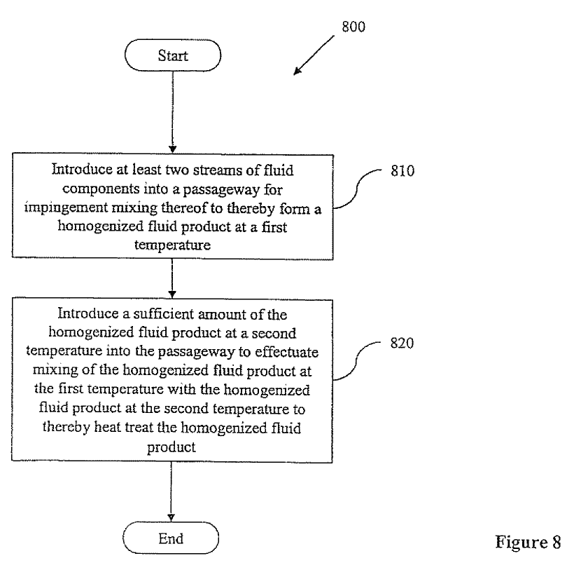

Illustrated in FIG. 8 is another embodiment of a methodology associated with heat treating a fluid product. With reference to FIG. 8, the process 800 includes introducing at least two streams of fluid components into a passageway for impingement mixing thereof to thereby form a fluid product at a first temperature (block 810). A sufficient amount of the fluid product at a second temperature can then be introduced the passageway to effectuate mixing of the fluid product at the first temperature with the fluid product at the second temperature to thereby heat treat the fluid product (block 820). In one embodiment, the second temperature can be less than the first temperature resulting in the cooling of the fluid product when the fluid product at a second temperature is mixed with the fluid product at the first temperature.

The present invention is further described by the following non-limiting example. The example is merely illustrative and does not in any way limit the scope of the present invention as described and claimed.

### EXAMPLE 1

Utilizing the system 600 illustrated in FIG. 6 and a circular orifice-type high shear mixing device 300 substantially similar to the one illustrated in FIG. 3 and described above, four experiments were conducted with water as the fluid stream at various flow rates. For all four experiments, the pressure differential in the orifice of the high shear mixing device was 15,000 psi and the input temperature (T1) of the water stream (Stream A) into the high shear mixing device was 20.7° C.

The results of the experiments are illustrated in Chart 1 below Mixing Zone represents the homogenized fluid product downstream from the orifice (i.e., in the high shear mixing zone) before such homogenized fluid product is mixed with Stream C The temperature of the homogenized fluid product in the Mixing Zone is indicated as T2. Stream C represents a water stream from a separate, cold water source. The temperature of Stream C is indicated as T3, Stream B represents the mixed water streams exiting the high shear mixing device 300 (i.e., Stream A and Stream C). The temperature of Stream B is indicated as T4.

CHART 1

| Mixing Zone | | Stream C | | Stream B | |
|---|---|---|---|---|---|
| Flow Rate | | Flow Rate | | Flow Rate | |
| (GPM) | T2 (° C.) | (GPM) | T3 (° C.) | (GPM) | T4 (° C.) |
| 1.2 | 46.9 | 1.8 | 12.9 | 3.0 | 25.3 |
| 1.2 | 45.8 | 1.2 | 13.5 | 2.4 | 27.5 |
| 1.2 | 46.6 | 0.9 | 13.7 | 2.1 | 29.9 |
| 1.2 | 46.7 | 0.6 | 13.5 | 1.8 | 32.2 |

While the present invention has been illustrated by the description of embodiments thereof, and while the embodiments have been described in considerable detail, it is not the intention of the applicants to restrict or in any way limit the scope of the appended claims to such detail. Additional advantages and modifications will readily appear to those skilled in the art. Therefore, the invention, in its broader aspects, is not limited to the specific details, the representative apparatus, and illustrative examples shown and described. Accordingly, departures may be made from such details without departing from the spirit or scope of the applicant's general inventive concept.

1. A method for heat treating a homogenized fluid product, the method comprising the steps of:
feeding a stream of fluid product ingredients under pressure through a local constriction of flow to effectuate high shear mixing of the fluid product ingredients in a high shear mixing zone downstream from the local constriction of flow and thereby form a homogenized fluid product at a first temperature; and
introducing a sufficient amount of the homogenized fluid product at a second temperature, which is less than the first temperature, into the high shear mixing zone to effectuate mixing of the homogenized fluid product at the first temperature with the homogenized fluid product at the second temperature to thereby heat treat the homogenized fluid product.

2. The method of claim 1 wherein the homogenized fluid product is selected from the group consisting of a pure liquid product, emulsion, suspensions, and liquid-gas dispersions.

3. The method of claim 1 wherein the second temperature is at least about 1° C. less than the first temperature.

4. The method of claim 1 wherein the second temperature is at least about 10° C. less than the first temperature.

5. The method of claim 1 wherein the second temperature is at least about 1% less than the first temperature.

6. The method of claim 1 further comprising the step of pre-mixing the fluid product ingredients prior to the feeding step.

7. The method of claim 1 wherein the homogenized fluid product at the first temperature has a concentration that is substantially equal to the concentration of the fluid product at the second temperature.

8. The method of claim 1 wherein the homogenized fluid product at the second temperature is supplied to the high shear mixing zone from a separate source of the homogenized fluid product at the second temperature.

9. A method for heat treating a homogenized fluid product, the method comprising the steps of:
feeding at least one stream of fluid product ingredients through a local constriction of flow to effectuate high shear mixing of the fluid product ingredients in a high shear mixing zone downstream from the local constriction of flow and thereby form a heated homogenized fluid product;
cooling at least a portion of the homogenized fluid product to thereby form a cooled homogenized fluid product; and
introducing the cooled homogenized fluid product into the high shear mixing zone to effectuate mixing of the homogenized fluid product with the cooled homogenized fluid product and heat treat the homogenized fluid product.

10. The method of claim 9 wherein the stream of fluid product ingredients includes a temperature sensitive material.

11. The method of claim 10 wherein the temperature sensitive material is selected from the group consisting of biological materials, organic materials, pharmaceutical materials, cellular materials, tissue materials, microbial materials, plant extracts, animal extracts, and certain food materials.

12. The method of claim 9 wherein the cooling step includes passing the portion of the homogenized fluid product through a cooling device to effectuate cooling of the homogenized fluid product.

13. The method of claim 9 wherein the temperature difference between the homogenized fluid product and the cooled homogenized fluid product is at least about 1° C.

14. A method for heat treating a homogenized fluid product, the method comprising the steps of:
feeding at least one stream of fluid product ingredients through a local constriction of flow to effectuate high shear mixing of the fluid product ingredients in a high shear mixing zone downstream from the local constriction of flow and thereby form a heated homogenized fluid product; and
mixing the heated homogenized fluid product and a cooling fluid in a flow-through channel downstream from a local constriction of flow provided in the flow-through channel to heat treat the homogenized fluid product, wherein the homogenized fluid product and the cooling fluid are substantially equal in composition.

15. The method of claim 14 further comprising the step of passing a stream of fluid product ingredients through the local constriction of flow to effectuate high shear mixing of the fluid product ingredients and thereby form the homogenized fluid product.

16. The method of claim 14 wherein the homogenized fluid product has a concentration that is substantially equal to the concentration of the cooling fluid.

17. The method of claim 14 wherein the mixing of the homogenized fluid product and the cooling fluid in the flow-through channel occurs at a sufficient distance downstream from the local constriction of flow to prevent damage to any temperature sensitive materials present in the homogenized fluid product.

18. A method for heat treating a fluid product, the method comprising the steps of:
introducing at least two streams of fluid components into a passageway for impingement mixing thereof to effectuate high shear mixing of the homogenized fluid product to thereby form a homogenized fluid product at a first temperature; and
introducing a sufficient amount of the homogenized fluid product at a second temperature into the passageway to effectuate high shear mixing of the homogenized fluid product at the first temperature with the homogenized fluid product at the second temperature to thereby heat treat the homogenized fluid product, wherein the second temperature is less than the first temperature.

19. A method for heat treating a homogenized fluid product, the method comprising the steps of:
introducing at least two streams of homogenized fluid product into a passageway for impingement mixing thereof to effectuate high shear mixing of the homogenized fluid product, wherein the temperature of one of the streams is substantially less than the temperature of the other stream to thereby heat treat the homogenized fluid product.

20-25. (canceled)
